(19) United States
(12) Patent Application Publication
(10) Pub. No.: US 2024/0141269 A1
(43) Pub. Date: May 2, 2024

(54) DEVICE FOR MODELLING A BLOOD LABYRINTH BARRIER

(71) Applicant: UNIVERSITÄT BASEL, Basel (CH)

(72) Inventors: Vesna PETKOVIC, Biel (CH); Marijana SEKULIC, Basel (CH)

(21) Appl. No.: 18/548,738

(22) PCT Filed: Mar. 8, 2022
(86) PCT No.: PCT/EP2022/055823

§ 371 Date: Sep. 1, 2023

(30) Foreign Application Priority Data
Mar. 8, 2021 (EP) 21161192.6

(51) Int. Cl. C12M 3/00; C12M 1/00; C12M 1/12; C12M 3/06

(52) U.S. Cl. CPC C12M 21/08; C12M 23/16; C12M 25/04; C12M 29/10

(57) ABSTRACT

A device is disclosed for modelling a blood labyrinth barrier of a human ear that includes a first fluid channel and a second fluid channel, and a membrane separating the first and second fluid channels. The membrane has a luminal side in the first fluid channel and an abluminal side in the second fluid channel, endothelial cells attached to the luminal side of the membrane, pericytes attached to the abluminal side of the membrane, and perivascular macrophage-type melanocytes arranged in the second fluid channel. A method of preparing such device is also disclosed. As well, a device with two fluid channels and a membrane as described above is disclosed, wherein the endothelial cells, pericytes and perivascular macrophage-type melanocytes are arranged in two fluid containers.

### TECHNICAL FIELD

The present invention relates to a device for modelling a blood labyrinth barrier of a human ear, and a method for preparing such device.

### BACKGROUND ART

Hearing impairment is a global health problem with a high socioeconomic impact, and it is associated with a high unmet medical need. Auditory hair cell damage in the inner ear due to aging, acoustic trauma, or exposure to antibiotics or chemotherapy underlies most cases of sensorineural hearing loss. Hearing loss is one of the 10 leading causes of disability globally. Unfortunately, there are no drug therapies that can protect or restore hearing.

Inner ear auditory hair cells and the blood labyrinth barrier, BLB, of a human ear are critical for normal hearing. The BLB comprises endothelial cells, ECs, pericytes, PCs, and perivascular macrophage like melanocytes, PVM/Ms, which are essential for maintaining BLB integrity. The BLB between the systemic circulation and stria vascularis is crucial for maintaining cochlear and vestibular homeostasis, facilitating nutrient and metabolite transport into the cochlea, and protecting the cochlea against inflammation and disease. BLB defects are associated with inner ear diseases that lead to hearing loss, including vascular malformations, Meniere's disease, and Alport syndrome.

Delivering therapeutics to the cochlea and vestibular system of the inner ear is complicated by their inaccessible location. It is particularly difficult to avoid adverse effects due to off-target binding, such as ototoxicity from antibiotics and chemotherapy. Systemic delivery of therapeutics to cochlea involves numerous challenges.

Therefore, in view of the complexity and inaccessibility of the inner ear there is a need for a blood labyrinth barrier model representing the blood labyrinth barrier of a human ear, thereby performing medical assessments and diagnostics as well as experiments thereon instead of the blood labyrinth barrier of the human ear or an animal. One of the medical assessments can be for instance the above-mentioned delivery of therapeutics to the cochlea and vestibular system of the inner ear.

### DISCLOSURE OF THE INVENTION

According to the invention this need is settled by a device, a kit and a method for manufacturing the device as defined by the features of the independent claims. Preferred embodiments are subject of the dependent claims. The device can be also called chip or microfluidic chip due to its form, structure and intended application.

In one aspect, the invention relates to a device for modelling a blood labyrinth barrier of a human ear. The device comprises a first fluid channel, a second fluid channel, and a membrane separating the first and second fluid channels. The membrane has a luminal side in the first fluid channel and an abluminal side in the second fluid channel. The device further comprises endothelial cells, pericytes and perivascular macrophage like melanocytes. Hereinafter the perivascular macrophage-like melanocytes may also be referred to as perivascular macrophage type melanocytes.

In one preferred embodiment, the endothelial cells are attached to the luminal side of the membrane, the pericytes are attached to the abluminal side of the membrane and the perivascular macrophage like melanocytes are arranged in the second fluid channel.

In another preferred embodiment, the device comprises a first container and a container arrangement, wherein the first container includes a first culture medium and the endothelial cells.

The container arrangement includes a second culture medium, pericytes and perivascular macrophage-type melanocytes. The container arrangement may comprise a second container including the second culture medium, the pericytes and the perivascular macrophage-type melanocytes. Or, the container arrangement of the kit may comprise a second container including the second culture medium and the pericytes, and a third container including a third culture medium and the perivascular macrophage-type melanocytes.

The device may be a microfluidic cell culture system that can incubate and/or culture the cells of the blood labyrinth barrier, thereby providing a blood labyrinth barrier model. The micro-engineered three-dimensional model of the blood labyrinth barrier cell culture can be used for the medical assessments and diagnostics as well as experiments. The progenitor cells can be taken from autopsy-derived adult human temporal bones. Using the blood labyrinth barrier model, it is possible to examine all important characteristics of cell phenotypes, which is helpful for successfully placing them on the device and establish a network with the flow.

The human ear's blood-labyrinth barrier (BLB) typically is composition of a network of capillary that form a structural and chemical barrier between the cochlea and systemic circulation. The vessels of the BLB are composed of specialized endothelial cells that lack fenestration, e.g., pores that allow rapid exchange of molecules between vessels and tissue, and that have extensive tight junctions that severely restrict cell permeability. Limited permeability restricts movement of substances from the systemic circulation to the interior of the cochlea which buffers the endolymph and perilymph from rapid changes in ionic or metabolic conditions. Limited BLB permeability also protects the auditory hair cells from exposure to molecules that are harmless to peripheral organs but toxic to hair cells in the cochlea. The BLB permeability is influenced by the extracellular matrix, pericytes, and perivascular macrophage-like melanocytes. These cells, along with the extracellular matrix, function as a spatially unit to regulate BLB permeability and maintain the integrity and homeostasis of the cochlea.

The term “fluid channel” may particularly relate to a micro-fluidic chamber, where the main portion of the chamber is in an elongated form that can be a substantially rectangular prism. The fluid channel may be filled or perfused with a culturing medium so that the cells can be incubated and cultured in the chamber.

The term “membrane” may particularly relate to a selective barrier that is formed of a thin foil and can be porous. The membrane can allow some molecules, ions or other small particles to pass through but can at the same time prevent the others. The membrane can be made of glass or polymer, e.g., polycarbonate or Polydimethylsiloxane, PDMS. Depending on the manufacturing process, the polycarbonate can be provided with a pore size of between about 0.4 μm and 1 μm and a pore density of 1.6×10⁶ cm⁻². Since the membrane is porous, the chemical or culture medium can pass though the membrane, and at the same time the cells can be in contact with the membrane that is attached to the membrane but not pass through the membrane.

According to the invention, the device not only comprises the inorganic components like the membrane but also the cells forming the blood labyrinth barrier model. Like this, the device is ready for specific uses in medical diagnostics or analysis, such as for screening for example ototoxicity of active pharmaceutical compounds, and other experiments in connection with the human ear's BLB. In particular, the BLB modelling structure of the device comprises the endothelial cells and the pericytes as well as the perivascular macrophage-type melanocytes such that the device is provided out of box and can efficiently and immediately be used for medical diagnostics or analysis and experiments.

In another aspect, the invention relates to a kit comprising a device for modelling a blood labyrinth barrier of a human ear, a first container and a container arrangement. The device of the kit has a first fluid channel and a second fluid channel, and a membrane separating the first and second fluid channels such that the membrane has a luminal side in the first fluid channel and an abluminal side in the second fluid channel. The first container includes a first culture medium and endothelial cells. The container arrangement includes a second culture medium, pericytes and perivascular macrophage-type melanocytes.

The kit according to the invention allows for efficiently providing all components required to put into operation a device according to the invention. More specifically, it allows for providing the cells in a proper manner such that they are not harmed before starting operation of the device. For example, after unpacking the kit, the cells cultured in the containers can be inserted to the device. In particular, the endothelial cells can be inserted into the first fluid channel, and the pericytes and perivascular macrophage-type melanocytes can be inserted into the second fluid channel, thereby forming the BLB simulating structure. Like this, the cells can securely survive particularly for a comparably long compared to being already attached to membrane.

In one preferred embodiment, the container arrangement of the kit comprises a second container including the second culture medium, the pericytes and the perivascular macrophage-type melanocytes. Such combined provision of the pericytes and the perivascular macrophage-type melanocytes allows for an efficient handling and seeding of the cells.

In another preferred embodiment, the container arrangement of the kit comprises a second container including the second culture medium and the pericytes, and a third container including a third culture medium and the perivascular macrophage-type melanocytes. Such separated provision of the pericytes and the perivascular macrophage-type melanocytes allows a separated seeding of the pericytes and inclusion of the perivascular macrophage-type melanocytes.

Any containers included in the kit preferably are hermetically sealed. Preferably, the cells are transported or provided frozen in dry ice within the containers. Such provisions of the cells inside the containers allows for protecting the cells before operation of the device.

In the following preferred embodiments of the device according to the invention and of the device of the kit according to the invention are described:

The membrane preferably has a permeability allowing a chemical transport between the first and the second fluid channel.

Preferably, the luminal side of the membrane is coated with collagen. Preferably, the abluminal side of the membrane is coated with fibrinogen. The coatings can enable a better attachment of the cells on the membrane and can also improve development of the cells.

Preferably, endothelial cells can be formed in a monolayer and can be attached to the luminal side of the membrane, pericytes can be formed in a monolayer and can be attached to the abluminal side of the membrane, and the perivascular macrophage-type melanocytes are cultured in the second fluid channel. The endothelial cells, the pericytes and the perivascular macrophage-type melanocytes form a blood labyrinth barrier model. The term “attached” in connection with “culturing” means that the cells are directly arranged or adhered on the luminal or the abluminal side of the membrane, and at the same time the cells can be cultured on the membrane in the fluid channel. The perivascular macrophage-type melanocytes can be also called perivascular-resident macrophage-like melanocytes.

Preferably, the device comprises a first inlet and a first outlet configured to selectively perfuse a first culture medium in the first fluid channel, and a second inlet and a second outlet configured to selectively perfuse a second culture medium in the first fluid channel. The culture mediums are used to incubate or culture the cells.

Advantageously, the endothelial cells are human endothelial cells, the pericytes are human pericytes and/or the perivascular macrophage like melanocytes are human perivascular macrophage like melanocytes. Including such human cells allows for efficiently modelling the human BLB.

Particularly when the endothelial cells are human endothelial cells, the device preferably includes the first culture medium, which comprises endothelial basal medium, fetal bovine serum, epidermal growth factor (recombinant human), basic fibroblast growth factor (recombinant human), insulin-like growth factor (long R3 IGF), vascular endothelial growth factor 165 (recombinant human), ascorbic acid, hydrocortisone, and penicillin/streptomycin.

Further, particularly when the pericytes are human pericytes, the device preferably includes the second culture medium, which comprises Dulbecco's modified eagle's medium (DMEM), fetal bovine serum, pericyte growth supplement, pigment epithelium-derived factor (PEDF—recombinant human), and penicillin/streptomycin solution.

Still further, particularly when the perivascular macrophage-type melanocytes are human perivascular macrophage-type melanocytes, the device preferably includes the second culture medium which comprises medium 254CF plus calcium chloride (CaCl₂)), fetal bovine serum, human melanocyte growth supplement, basic fibroblast growth factor (recombinant human), insulin (recombinant human), hydrocortisone, and gentamicin/amphotericin B.

Preferably, the device comprises a first and a second cover sheet, a first medical tape having at least one first opening portion, wherein the first medical tape adhering at the first cover sheet, thereby forming the first fluid channel, and a second medical tape having at least one second opening portion, wherein the second medical tape adhering at the second cover sheet, thereby forming the second fluid channel. In a cross-section view, the medical tape can be seen as the walls for the fluid channel. Thus, the first opening portion forms the first fluid channel and the second opening portion forms the first second channel. The first cover sheet forms the top of the first fluid channel, the second cover sheet forms the bottom of the second fluid channel, and the membrane separates the first and second fluid channel.

The adhesive used in the medical tapes can be any adhesive suitable to provide appropriate adhesion, such as a silicone-based adhesive. However, preferably, each of the first medical tape and the second medical tape comprises an acrylic-based adhesive. Such adhesive can be particularly beneficial since it shows comparably high resistance to moisture, solvents and chemicals. Furthermore, it can sustain comparably drastic temperature changes. Like this, a solid connection between the medical tapes, the membrane and the cover sheets can be achieved which may endure the lifecycle of the device.

Further, each of the first cover sheet and the second cover sheet preferably comprises of or consists of glass and/or of polycarbonate (PC). Compared to cover sheets of other materials, e.g., of polydimethylsiloxane (PDMS), glass and PC cover sheets allow a particularly appropriate adhesion. More specifically, when medical tapes having acrylic-based adhesives are used, a comparably high adhesion and, thus, strong connection between the tapes and the cover sheets can be achieved of acrylic to glass is high. However, the adhesive strength to PDMS is not sufficient.

Still further, each of the first medical tape and the second medical tape comprising the acrylic-based adhesive can be laminated with a silicone-based adhesive. Such laminated adhesive allows a to increase the adhesion strength to cover sheets made of materials different from glass or PC such as, in particular, to cover sheets made of PDMS.

Preferably, the first medical tape comprises a plurality of the first opening portions, and the second medical tape comprises a plurality of the second opening portions. Accordingly, in this embodiment, the device has a multiple first channels and a multiple second channels.

The opening portion comprises a main portion in an elongated form, for instance, a substantially rectangular prism which can be seen as slot-shaped or as a rectangular since the medical tape is thin. In three-dimension, the main portion of the opening portion is in fact in form of an elongated prism or rectangular prism with a comparatively small height, from a side view. The form of the opening portion of the medical tape corresponds to the form of the fluid channel since the opening portion forms the fluid channel.

Preferably, the first fluid channel comprises: a first main portion in form of a rectangle prism, and a first incoming portion connected with the first inlet and a first outgoing portion connected with the first outlet, and preferably the second fluid channel comprises: a second main portion in form of a rectangle prism, and a second incoming portion connected with the second inlet and an outgoing portion connected with the second outlet. In general, the form of the fluid channel is defined by the opening of the medical tape.

In particular, the main portion, i.e., the first or the second fluid channel, is used for incubating or culturing the cells, and the incoming and outgoing portions are used for creating the fluidic within the main portion. The inlet and the outlet can be mounted on a top side of the first cover sheet. The culturing medium, i.e. the culture medium, can be supplied though the inlet towards the main portion of the opening, i.e., the fluid channel, and leaves the opening at the outgoing portion.

Preferably, the first incoming portion does not overlap with the second incoming portion from a view perpendicular to the medical tape, and the second incoming portion does not overlap with the first main portion from the view perpendicular to the medical tape. Preferably, the first outgoing portion does not overlap with the second outgoing portion from the view perpendicular to the medical tape, and the second outgoing portion does not overlap with the first main portion from the view perpendicular to the medical tape. Accordingly, the first and second inlet can be arranged on the same cover sheet and supply the culture medium to the first and the second incoming portion, respectively.

Preferably, the device comprises a first electrode arranged in the first fluid channel and a second electrode in the second fluid channel. Preferably, the device further comprises a measurement unit configured to measure a potential between the first and the second electrodes, thereby determining an integrity of the modelled blood labyrinth barrier. The integrity can be used for determining an integrity of the modelled blood labyrinth barrier which represents the permeability of the blood labyrinth barrier of human ear. This is particularly interesting in examination of the penetration of the therapeutics agent through the blood labyrinth barrier of the human ear, using the blood labyrinth barrier model.

Alternatively or additionally, the device can further comprise a detection unit configured to detect an entry of a therapeutic agent through the blood labyrinth barrier model, e.g. how long does it take until the therapeutic agent penetrates the model and at the speed the therapeutic agent penetrates the model as well as the amount of therapeutic agent can penetrate the blood labyrinth barrier model. Accordingly, the delivery the therapeutic agent through the blood labyrinth barrier of the human ear can be examined.

Preferably, the device comprises or is connected with a control unit that is configured to control a pressure applied to the device for simulating a disease such as inflammation and Meniere's disease using the blood labyrinth barrier model. In this case the external pressure may be applied to the cover sheet of the device, thereby simulating the human blood labyrinth barrier under the disease. This enables the medical diagnostics, analysis and experiments using the modelled blood labyrinth barrier instead of the human or animal blood labyrinth barrier, i.e. the modelled blood labyrinth barrier mimicks the human or animal blood labyrinth barrier. In other words, cells in the device be set up as described above. Upon arrangement or attachment of the cells in the fluid channels, the cells can be treated with the culture medium that can contain various inflammatory factors, thereby contributing to the weakened integrity of the barrier. After about three days, additional steers can be caused with constant pressure on the cells, which mimics the situation as with hydrops.

The first and second fluid channels may have a length of about 16 millimeter (mm) to about 21 mm and a width of about 0.6 mm to about 1.5 mm. Fluid channels of such dimension allow to use a comparably small number of cells, which allows to apply a wider range of different processes and testing more drug compounds. Further, it allows the cells proliferate in a comparably short time and to reach a confluence in this respect comparably early to begin the validation of the BLB, which may shorten the time of screening and optimization of new treatment.

A pressure inside of the first and second fluid channels may be adapted to more or less correspond to the pressure inside the labyrinth of the human ear. For this purpose, the device may comprise a pump such as a peristaltic pump allowing a sufficiently accurate adjustment of the pressure.

In another aspect, the present invention provides a method for manufacturing for modelling a blood labyrinth barrier of a human ear. The method comprises steps of cutting at least one first and one second opening in form of a rectangle prism in a first and a second medical tape using a layout mask, respectively, patterning a membrane using the layout mask, preparing a first and a second cover sheet using the layout mask, assembling the device, and curing and sterilise the device. The step of assembling comprises steps of (i) adhering the first cover sheet with the first medical tape having the at least one first opening portion, thereby forming a first fluid channel, (ii) adhering the second cover sheet with the second medical tape having the at least one second opening portion, thereby forming a second fluid channel, (iii) adhering the membrane with the first medical tape and the second medical tape.

The layout mask preferably includes electronic layouts for the first and second medical tapes, the membrane, and the first and second cover sheet. The layout mask can be prepared using a computer. The electronic layouts are data sets that are either defined using specified text format or they can be visual presentations such as drawings. The machine used to prepare the components such as medical tapes and the cover sheets as well as the membrane can understand the format of electronic layouts and executes the manufacturing processes on the respective components.

Each of the openings can comprise a main portion in form of an elongated rectangle prism, an incoming portion being fluidic connected with the inlet and an outgoing portion being fluidic connected with the outlet. The inlet and the outlet of each opening can be mounted on the first cover sheet. In particular, the openings may have a length of about 16 mm to about 21 mm and a width of about 0.6 mm to about 1.5 mm.

Preferably, the membrane is patterned using a laser. For later assembly of the multilayer microfluidic tape device or chip as well as for not hindering the flow to the bottom compartments, the polycarbonate membrane may need to be patterned. The small-scale feature size necessary for patterning the membrane made the utilization of the cutting plotter unapproachable. Therefore, the membrane is preferably be patterned with a laser cutter.

Preferably, the method comprises steps of perfusing a first and a second culture medium in the first and the second fluid channel respectively. The first culture medium comprises progenitor endothelial cells, and the second culture medium comprises progenitor cells for pericytes and perivascular macrophage-type melanocytes.

Advantageously, the progenitor endothelial cells are human progenitor endothelial cells. Thereby, the first culture medium may comprise endothelial basal medium, fetal bovine serum, epidermal growth factor, basic fibroblast growth factor, insulin-like growth factor, vascular endothelial growth factor, ascorbic acid, hydrocortisone, and penicillin/streptomycin.

Further, advantageously, progenitor cells for pericytes are human progenitor cells for pericytes. Thereby, the second culture medium may comprise Dulbecco's modified eagle's medium, fetal bovine serum, pericyte growth supplement, pigment epithelium-derived factor, and penicillin/streptomycin solution.

Still further, advantageously, the perivascular macrophage-type melanocytes are human perivascular macrophage-type melanocytes. Thereby, the second culture medium may comprise medium 254CF plus calcium chloride, fetal bovine serum, human melanocyte growth supplement, basic fibroblast growth factor, insulin, hydrocortisone, and gentamicin/amphotericin B.

Preferably, each of the first medical tape and the second medical tape used in the method comprises an acrylic-based adhesive. Further, each of the first cover sheet and the second cover sheet preferably comprises of glass and/or of polycarbonate.

Preferably, the device for modelling a blood labyrinth barrier of a human ear involved in the kit is a device according to the invention or a preferred embodiment thereof as described above or below.

The device enables generation of a model of 3D cultures of the blood labyrinth barrier that is located in the stria vascularis of the cochlea in the inner ear. Their function is the omission of substances such as nutrients and ions needed for the normal function of hearing, and homeostasis of the cochlear potential but also of medicaments and ototoxic substances.

In general, the device provides tools for investigating specific contributions of the blood labyrinth barrier to physiological and pathophysiological mechanisms underlying hearing loss. This technology also presents a promising alternative to reduce animal testing. The model blood labyrinth barrier model created by the device can be applied for varies applications such as: studying blood labyrinth barrier integrity and band transport; use of barrier components generated from human cochlea (novel) to capture blood labyrinth barrier properties; use of classical and well accepted validation models as Transepithelial or Transendothelial Electrical Resistance, TEER; permeability measurements to improve characterization of blood labyrinth barrier properties; recognizing the mechanism of entry of nutritious, harmful as well as therapeutic components or the like, i.e. anything that comes with systemic circulation, across the blood labyrinth barrier; selective delivery of harmful compounds from nutritious or the like, across the blood labyrinth barrier; and discover how barrier component cells interact in inflammation and Meniere's disease within a second blood labyrinth barrier model that can mimic those diseases. Ultimately, the device can establish a stable chip model to discover new therapies for patients and breakthrough innovations in the field of inner ear research. The selective delivery can avoid the entry of haemostatics into the cochlea during chemotherapy since the haemostatics in a short time can lead to hearing loss.

According to the present invention the device can be made with low-cost material. The microfabrication strategy according to the present invention uses low-cost materials and methods such as biomedical adhesive tape and a cutting plotter for rapid, economical, and high throughput organ-on-a-chip fabrication. In comparison, the conventional microfabrication approaches like photo and soft lithography determine the fabrication of these systems with somewhat suboptimal materials, lengthy manufacturing processes and the need for high resource microfabrication environments.

The device fabricated according to the present invention is comparably inexpensive but far more generous in the output information.

In addition, the present invention can establish a 3D culture of blood labyrinth barrier on the chip derived first from the mouse and then human cochlea.

In the conventional approach, in order to study the molecular mechanisms underlying the function of the barrier under physiological conditions, a large number of animals can be used and time-consuming experiments need to be carried out. In contract, the device according to the present invention provides an organ-on-chip system offering a platform that (i) requires a small number of cells, (ii) provides quick results, and (iii) is more accurate and physiologically relevant. Overall, the current in vitro platform could refine in vivo experiments and reduce the number of animals used in the studies. In addition to an in vitro instrument for pharmacological studies, the proposed microfluidic cell culture system of the blood labyrinth barrier can be used to answer basic questions about the development and progression of degenerative disease mechanisms in the inner ear. It is not only a valuable tool for laboratories doing basic research in related fields but also can be useful for broader pharma drug discovery and toxicity studies.

### BRIEF DESCRIPTION OF THE DRAWINGS

The device and method according to the invention are described in more detail hereinbelow by way of an exemplary embodiment and with reference to the attached drawings, in which:

FIG. 1 shows an exemplary embodiment of the device according to the present invention;

FIG. 2 shows an explosive view of the device according to the present invention;

FIG. 3 shows the first and the second medical tapes according to the present invention;

FIG. 4 shows an explosive view of the device with a plurality of fluid channel according to the present invention;

FIG. 5 shows a plurality of the fluid channels of the device according to the present invention;

FIG. 6 shows a simulation of the inflammation and Meniere's disease using the blood labyrinth modelled by the device according to the present invention;

FIG. 7 shows an exemplary manufacturing process according to the present invention; and

FIG. 8 shows a schematic of the BLB network and cell extraction and purification of total RNA or protein lysate from a microfluidic device, wherein the central membrane region contains a network of BLB cells.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

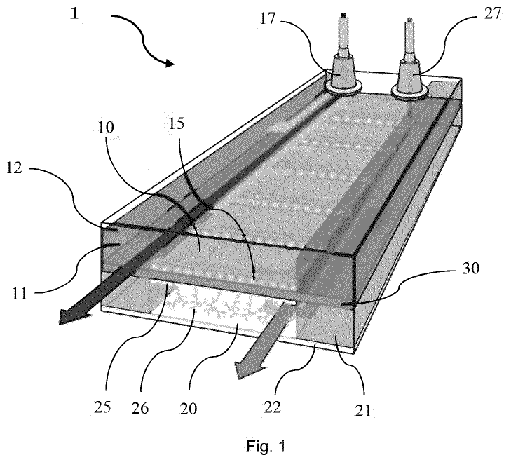

FIG. 1 shows an exemplary embodiment of the device 1 according to the present invention, where the device 1 comprises a first fluid channel 10, a second first fluid channel 20 and a membrane separating the first and the second fluid channels.

A first medical tape 11 forms the walls from left and right side of the first fluid channel 10. A first cover sheet 12 made of glass or polymer covers the first fluid channel from the top. The membrane 30 serves as a base for the first fluid channel. In other words, the first fluid channel 10 is enclosed by the cover sheet 12 and the first medical tape 11 as well as the membrane 30. An inlet 17 is connected with the first fluid channel 10 and a first culturing medium can be supplied therethrough.

Similarly, a second medical tape 21 forms the walls from left and right side of the second fluid channel 20. A second cover sheet 22 made of glass or polymer covers the first fluid channel from the bottom. The membrane 30 serves as a top for the second fluid channel 20. In other words, the second fluid channel 20 is enclosed by the cover sheet 22 and the second medical tape 21 as well as the membrane 30. An inlet 27 is connected with the second fluid channel 20 and a second culturing medium can be supplied therethrough.

In an exemplary embodiment, the device may comprise the first and the second culturing medium. In a further exemplary embodiment, the device may comprise the first and the second culturing medium as well as the blood labyrinth barrier model including the endothelial cells 15 attached to the luminal side of the membrane 30 in the first fluid channel 10, the pericytes 25 attached to the abluminal side of the membrane 30 in second first fluid channel 20, and the perivascular macrophage-type melanocytes 26 in the second fluid channel 20.

The membrane 30 has a luminal side in the first fluid channel 10. In other words, the upper side of the membrane 30 is the luminal side. The endothelial cells 15 can be incubated in the first fluid channel 10, in particular, the endothelial cells 15 are attached on the luminal side of the membrane 30. The pericytes 25 can be incubated in the second fluid channel 20, in particular, the pericytes 25 are attached on the abluminal side of the membrane 30. Furthermore, the perivascular macrophage-type melanocytes 26 are incubated in the second fluid channel 20.

Accordingly, a blood labyrinth barrier model is formed by the endothelial cells 15, the pericytes 25 and perivascular macrophage-type melanocytes 26. This blood labyrinth barrier model is cultured in the device and can be used for medical diagnostics and experiments including disease modelling or test for delivering therapeutics to the cochlea and vestibular system.

The progenitor cells for the endothelial cells 15, the pericytes 25 and perivascular macrophage-type melanocytes 26 can be taken from autopsy derived human temporal bones or the mouse blood labyrinth barrier cells.

Using cell sorting and antibody markers, the pure single cell type cultures can be selected. Furthermore, the blood labyrinth barrier cells could be co-cultured on an porous membrane cell culture inserts and they expressed markers of tight junctions. In particular, the endothelial cells grew on the apical surfaces, and perivascular macrophage-type melanocytes grew on the basal surfaces of the porous membranes. After 5 days of growth in vitro, the cells can be stained with specific markers, e.g., von Willebrand factor, vWF, for the endothelial cells, platelet-derived growth factor β, PDGFRβ, for pericytes and F4/80 for the perivascular macrophage-type melanocytes cells. Like this, the cells can be identified if they indeed are the expected type of cells. The perivascular macrophage-type melanocytes crossed the pores of the porous membrane and formed connections with the endothelial cells. The quantitative real time polymerase chain reaction, PCR or qPCR, can be performed to explore the expression of tight junction genes in co-cultured the endothelial cells and the perivascular macrophage-type melanocytes. The qPCR results can indicate the expression of cell-type specific genes. These cells expressed the majority of genes specific for the tight junctions found in the blood labyrinth barrier model. These results suggested that the co-cultured cells could build a natural barrier with typical blood labyrinth barrier properties.

As explained above the device comprises a first electrode arranged in the first fluid channel and a second electrode in the second fluid channel (not shown in FIG. 1). The electrodes can be used for measurement of a potential between the first and the second electrodes, thereby determining a permeability integrity of the modelled blood labyrinth barrier which represents the permeability of the blood labyrinth barrier of human ear.

In the following the present invention merely focusses on one electrode for explanation of the potential measurement. The TEER is a widely accepted quantitative technique to measure the integrity of tight junction dynamics in cell culture models of endothelial and epithelial monolayers. To successfully treat certain diseases of organs protected by physiological barriers, it is necessary to develop methods that can enable the transport of therapeutic drugs across these barriers in order to reach the target tissue. BLB functions and the quality of the barrier rely on the synergism between different BLB cell types. In the BLB chip with integrated electrode, the barrier efficiency will be evaluated by TEER. The device with integrated electrodes according to the present invention can enable real-time, non-invasive monitoring of TEER. For real-time data collection during experiments the EVOM2 will connected to LabView on a PC via a data acquisition device.

The TEER measurements can be carried out using a 4-point impedance measurement over the frequency range 100 kHz to 10 Hz and recorded at days 1, 4, 6, and 12 post-seeding. Over the course of BLB experiments, TEER was measured twice a day to monitor cell confluence and development of tight junctions. Impedance and capacitance values will be determined for each time point. To calculate TEER, the measurements from the chips in the absence of the cells were subtracted from the resistance of each device, and then the values were multiplied by the surface area of endothelial monolayer overlapping with the lower channel. Background resistances R_{b} were subtracted from total resistance R_{C} at each time point and normalized for area, giving TEER values in om cm 2 as in the following equation: TEER=(R_{c}−R_{b}).

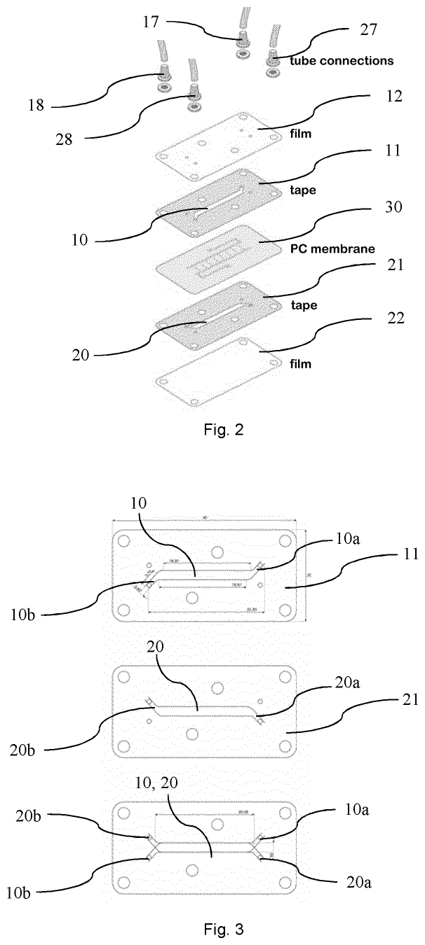

FIG. 2 shows an explosive view of the device 1 according to the present invention. The device comprises a first inlet 17, a first outlet 18, a second inlet 27, and a second outlet 28 for delivery of culturing mediums into the fluid channels 10, 20. The inlets 17, and the outlets 18, 28 can be mounted on the first cover sheet 12.

The first medical tape 11 has at least one first opening 10. The first medical tape is doubled sided adhesive. Thus, it can adhere on the one side with the membrane 30 and on the other side with the first cover sheet 12. When the first medical tape 11 is adhered to bottom side of the first cover sheet and the top side of the membrane 30, the opening 10 forms the first fluid channel 10.

Similarly, the second medical tape 21 has at least one second opening 20. The second medical tape is doubled sided adhesive. Thus, it can adhere on the one side with the membrane 30 and on the other side with the second cover sheet 22. When the second medical tape 21 is adhered to the top side of the second cover sheet 22 and bottom side of the membrane 30, the opening 20 forms the second fluid channel 20.

A plurality of holes is provided to the cover sheets 12, 22, the medical tapes 11, 21, and the membrane 30, for passing through the culturing mediums.

When the components of the device are assembled together, as explained before the openings 10, 20 of the first and second medical tapes 11, 21 form the first and second fluid channels 10, 20 respectively. In particular, a fluidic, i.e., the first culturing medium, can be perfused from the first inlet 17 through one of the plurality holes on corner of the first cover sheet 12 into the first fluid channel 10 and leave at the first outlet 18, thereby culturing the endothelial cells 15. Another fluidic, i.e., the second culturing medium, can be perfused from the second inlet 27 through one of the plurality holes on the first cover sheet 12 and first medical tapes as well as on the membrane 30 into the second fluid channel 20 and finally leave at the second outlet 28, thereby culturing the pericytes 25 and the perivascular macrophage-type melanocytes 26.

FIG. 3 shows the first and the second medical tapes according to the present invention in more detail. In particular, the first medical tape 11 has a main portion 10 which is in form of a rectangular from the top view, as well as a first incoming port 10a that can be connected to the first inlet 17 and a first outgoing portion 10b that can be connected to the first outlet 18. Similarly, the second medical tape 11 has a main portion 20 which is in form of a rectangular from the top view, as well as a second incoming port 20a that can be connected to the second inlet 27 and a second outgoing portion 20b that can be connected to the second outlet 28.

As shown, the first incoming portion 17 and the second incoming portion 27 are bent in different directions, i.e., from the top view there have different angles in respect to the longitudinal middle line of the main portion. This enables that the first and second inlets can be arranged on the same cover sheet, i.e., the first cover sheet 12. As explained above, the first culturing medium goes through the holes of first sheet 12, first medical tape 11 and the membrane 30 into the first fluid channel 10. The second culturing medium has a bit longer way to reach the second fluid channel, i.e., it goes through the holes of first sheet 12, first medical tape 11 and the membrane 30 into the second fluid channel 20. The similar configuration applies to the first outgoing portion 10b of the first opening 10 and the second outgoing portion 20b of the second opening 20. The bottom drawing of FIG. 3 illustrates a top view of the first and second medical tape 11, 21 stacked together.

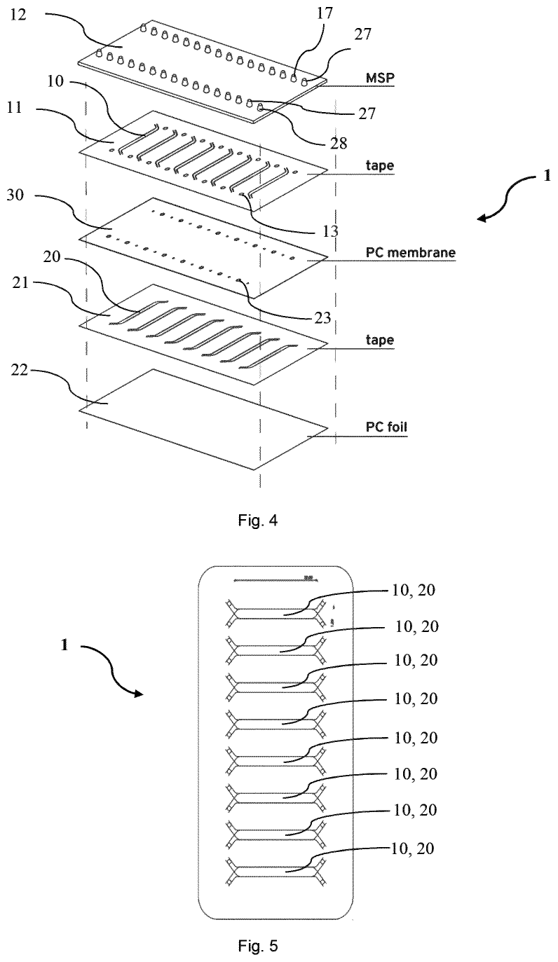

FIG. 4 shows an explosive view of the device with a plurality of the fluid channels, in particular, eight first fluid channels and eight second fluid channels, according to the present invention. Similar as shown in FIG. 2, the device 1 comprises a plurality of the first inlets 17, a plurality of the first outlets 18, a plurality of the second inlets 27, and a plurality of the second outlets 28 for delivery of culturing mediums into a plurality of the fluid channels 10, 20. The inlets 17, 27 and the outlets 18, 28 can be mounted on the first cover sheet 12.

The first medical tape 11 has a plurality of the first openings 10. The first medical tape is doubled sided adhesive. Thus, it can adhere on the one side with the membrane 30 and on the other side with the first cover sheet 12. When the first medical tape 11 is adhered to bottom side of the first cover sheet and the top side of the membrane 30, the openings 10 form the plurality of the first fluid channels 10.

Similarly, the second medical tape 21 has a plurality of the second openings 20. The second medical tape is doubled sided adhesive. Thus, it can adhere on the one side with the membrane 30 and on the other side with the second cover sheet 22. When the second medical tape 21 is adhered to the top side of the second cover sheet 22 and bottom side of the membrane 30, the openings 20 form the plurality of the second fluid channels 20.

A plurality of holes 13, 33, are provided to the cover sheets 12, 22 (holes not shown), the first medical tape 11 (holes shown as 13), the second medical tape 21 (holes not shown), and the membrane 30 (holes shown as 33), for passing through the culturing mediums.

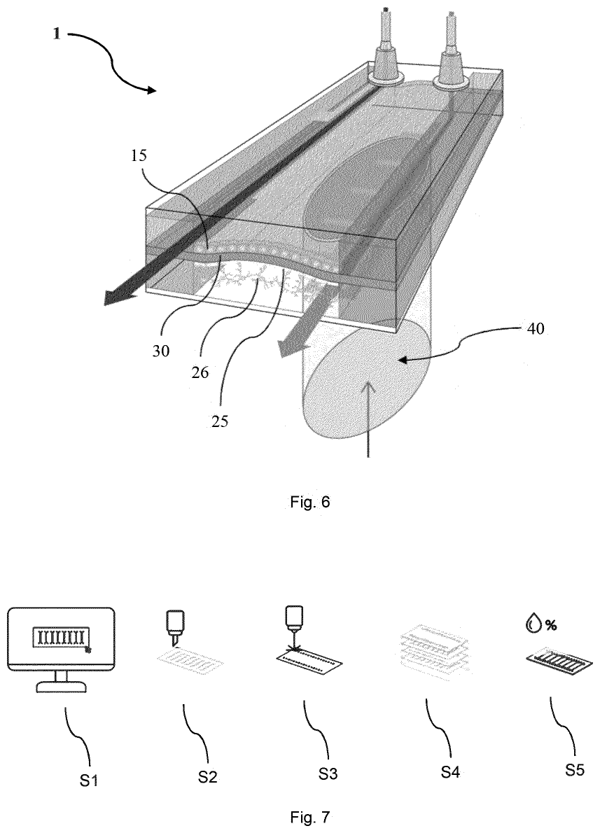

FIG. 5 shows a device 1 with a plurality of the first and second fluid channels 10, 20 each having an incoming and an outgoing portion. In this configuration, the device can include a plurality of the blood labyrinth barrier models, each of them can be used for a medical diagnostic and experiment.

The process of culturing and incubating the blood labyrinth barrier model using the device can be as follows:

The progenitor cells, which may be mouse progenitor cells, initially are placed into the fluid channels. In particular, the progenitor endothelial cells are attached onto the luminal side of the membrane, the progenitor pericytes are attached onto the abluminal side of the membrane, and the progenitor perivascular macrophage-type melanocytes are arranged in the second fluid channel. After the attachment on the surface of the membrane and the arrangement in the fluid channel, the fluid channels can be connected to a pump that will deliver the culture medium.

For hosting cells, the device should be sterilizable, biocompatible, and stable under applied flow and at the standard cell culture temperature of about 37° C. for extended periods of time. Therefore, flow-through experiments and optical evaluation of the device built with the different type of tape can be performed. To assess the fabricated device in terms of stability and leakage for later usage in cell assays, the fluid channels can be exposed to about 70% ethanol for 10 min prior to flow-through experiments with water.

Then endothelial cells can be seeded at 1×10⁷ cells ml⁻¹ density into the center of the first channel and incubated for 6 hours to allow adhesion of cells onto the collagen-coated polycarbonate membrane. Prior to seeding the pericytes into the center channel of the lower layer, the second fluid channel will be coated with 50 μg ml⁻¹ fibronectin for 1 h at 37° C. while the device can be placed upside down. Then 1×10⁶ perivascular macrophage-type melanocytes suspended in a 100 μl of Matrigel solution can be seeded into the same channel that pericytes are cultured.

The final concentration of Matrigel can be calculated to be 5 mg ml⁻¹. After gelation of Matrigel in the channel by incubating at about 37° C. for about 30 min, the cell culture medium can be filled into the two side channels to avoid the gel drying out. The final cell number ratio between endothelial cells and pericytes in a device can be 1.5:1, and the ratio between endothelial cells and perivascular macrophage-type melanocytes can be about 2:1, which can be optimized for PVMs to cover about 99% of the perivascular surface of the endothelium.

After 24 hours of culture to stabilize cells in the microfluidic device, the first channel can be connected to a peristaltic pump with 8 or 16 channels with the flow rate of 16 μL min-1 to give cells the shear stress of 4 dyne cm-2, which corresponds to the shear stress levels in the cochlea. The pump is connected via additional tubing to channel outlets and elevated syringe reservoirs connected by additional tubing to the inlets.

The culture mediums suitable for culturing mouse cells can be composited using the two basic mediums as follows:

| Basic medium BM1 (for 100 ml) | Basic medium BM2 (for 100 ml) |
|---|---|
| DMEM/high-glucose/ | DMEM/high-glucose/ |
| F12 (mixed 1:1) | F12 (mixed 1:1) |
| EGF (20 ng/ml)-80 μl | EGF (20 ng/ml)-80 μl |
| bFGF (10 ng/ml)-10 μl | bFGF (10 ng/ml)-10 μl |
| IGF-1 (50 ng/ml)-5 μl | IGF-1 (50 ng/ml)-5 μl |
| 0.5 ml penicillin | 200 μl gentamicin/ |
| streptomycin solution | amphotericin B solution |
| 2 ml human serum | 2 ml human serum |
| Filter through a 0.22 μm filter | Filter through a 0.22 μm filter |

Endothelial cells culture medium, i.e. EC medium (for 100 ml)
100 μl ECGF
99 ml BM1
ECGF (sigma E0760) preparation: 1
5 μg in 5 ml sterile balanced salt solution)
Pericytes culture medium, i.e. PC medium (for 100 ml)
25 μl PEDF
100 ml BM1
Perivascular macrophage-type melanocytes culture
medium, i.e. PVM medium (for 100 ml)
1 ml HMGS
99 ml BM2

The three mediums, i.e. EC medium, PC medium and PVM medium, firstly serve to develop cells in monocultures. When using the microfluid device according to the present invention, the EC medium will be added in the first channel. After about 24 hours when EC attach to the membrane, the PC together with the PC medium can be inserted and maintained in the second channel. After about another 24 hours, the PVM in matrix or hydrogel can be added into the second channel as well. Then, the PC and VM medium in the ratio of about 50:50 can be added into the second channel. In other words, the first culture medium can be the EC medium and the second can be a mixture comprising about 50% PC medium and about 50% PVM medium.

FIG. 6 shows a simulation of the inflammation and Meniere's disease using the blood labyrinth modelled by the device according to the present invention. In particular, a pressure 40 is controllable applied to the device. Consequently, the membrane 30 of the device is deformed, thereby the blood labyrinth barrier model 15, 25, 26 cultured in the device is deformed and can simulate a blood labyrinth barrier under the disease.

FIG. 7 shows an exemplary manufacturing process according to the present invention. Firstly, an electronic layout mask can be defined using a computer, S1, wherein the electronic layout mask may be defined as drawings for the different components, i.e., the cover sheets, the membrane and the medical tapes. This step usually takes about 10 to 40 minutes. Secondly, the openings on the medical tapes are cut off by a computer-controlled machine using the electronic layout mask, S2, which takes about 10 minutes. Thirdly, the membrane is patterned with a laser machine using the electronic layout mask, S3, which takes about 10 to 15 minutes. Then, the prepared components are stacked on each other, S4, which takes about 10 to 16 minutes. Finally, the device is cured and sterilised, S5, which takes about 20 minutes to 2 hours.

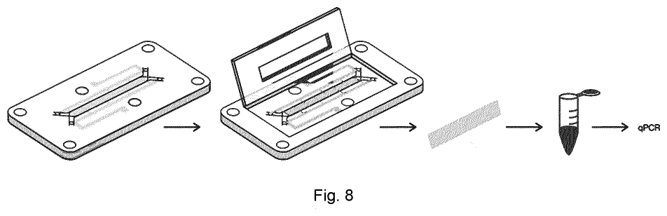

FIG. 8 shows a schematic of the BLB network and cell extraction and purification of total RNA or protein lysate from a microfluidic device. The BLB model including the cells are attached to the membrane of the device. The membrane can be taken out from the device and then delivered the qPCR test. As discussed above, the qPCR result indicates the expression of cell-type specific genes, thereby the property and characteristic of the BLB model can be determined by the qPCR.

This description and the accompanying drawings that illustrate aspects and embodiments of the present invention should not be taken as limiting—the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the FIGS. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A device for modelling a blood labyrinth barrier of a human ear, comprising:
a first fluid channel;
a second fluid channel; and
a membrane separating the first and second fluid channels,
wherein the membrane has a luminal side in the first fluid channel and an abluminal side in the second fluid channel,
wherein endothelial cells are attached to the luminal side of the membrane,
wherein pericytes are attached to the abluminal side of the membrane, and
wherein perivascular macrophage-type melanocytes are arranged in the second fluid channel.

2. The device according to claim 1, wherein the membrane has a permeability allowing a chemical transport between the first fluid channel and the second fluid channel.

3. The device according to claim 1, wherein the luminal side of the membrane is coated with collagen and/or the abluminal side of the membrane is coated with fibrinogen.

4. The device according to claim 1, comprising a first inlet and a first outlet configured to perfuse a first culture medium through the first fluid channel, and a second inlet and a second outlet configured to perfuse a second culture medium through the first fluid channel.

5. The device according to claim 4, including the first culture medium, wherein the first culture medium comprises endothelial basal medium, fetal bovine serum, epidermal growth factor, basic fibroblast growth factor, insulin-like growth factor, vascular endothelial growth factor, ascorbic acid, hydrocortisone, and penicillin/streptomycin.

6. The device according to claim 5, including the second culture medium, wherein the second culture medium comprises Dulbecco's modified eagle's medium, fetal bovine serum, pericyte growth supplement, pigment epithelium-derived factor, and penicillin/streptomycin solution.

7. The device according to claim 5, including the second culture medium, wherein the second culture medium comprises medium 254CF plus calcium chloride, fetal bovine serum, human melanocyte growth supplement, basic fibroblast growth factor, insulin, hydrocortisone, and gentamicin/amphotericin B.

8. The device according to claim 1, comprising:
a first cover sheet and a second cover sheet;
a first medical tape having at least one first opening portion, wherein the first medical tape adheres at the first cover sheet, thereby forming the first fluid channel; and
a second medical tape having at least one second opening portion, wherein the second medical tape adheres at the second cover sheet, thereby forming the second fluid channel.

9. The device according to claim 8, wherein each of the first medical tape and the second medical tape comprises an acrylic-based adhesive.

10. The device according to claim 8, wherein each of the first cover sheet and the second cover sheet comprises glass and/or polycarbonate.

11. The device according to claim 8,
wherein the first fluid channel comprises a first main portion in an elongated form, and a first incoming portion connected with the first inlet and a first outgoing portion connected with the first outlet, and
wherein the second fluid channel comprises a second main portion in an elongated form, and a second incoming portion connected with the second inlet and an outgoing portion connected with the second outlet.

12. The device according to claim 11,
wherein the first incoming portion does not overlap with the second incoming portion in a direction perpendicular to the first medical tape, and the second incoming portion does not overlap with the first main portion in the direction perpendicular to the second medical tape, and
wherein the first outgoing portion does not overlap with the second outgoing portion in the direction perpendicular to the first medical tape, and the second outgoing portion does not overlap with the first main portion in the direction perpendicular to the second medical tape.

13. The device according to claim 1, comprising a first electrode arranged in the first fluid channel and a second electrode in the second fluid channel.

14. The device according to claim 13, comprising a measurement unit configured to measure a potential between the first electrode and the second electrode, thereby determining a permeability of the modelled blood labyrinth barrier.

15. The device according to claim 1, comprising a control unit configured to control a pressure applied to the device for simulating a disease such as inflammation and Meniere's disease using the blood labyrinth barrier modelled by the device.

16. A kit comprising:
a device for modelling a blood labyrinth barrier of a human ear;
a first container; and
a container arrangement,
wherein the device has a first fluid channel and a second fluid channel, and a membrane separating the first and second fluid channels such that the membrane has a luminal side in the first fluid channel and an abluminal side in the second fluid channel,
wherein the first container includes a first culture medium and endothelial cells, and
wherein the container arrangement includes a second culture medium, pericytes and perivascular macrophage-type melanocytes.

17. The kit of claim 16, wherein the container arrangement comprises a second container including the second culture medium, the pericytes and the perivascular macrophage-type melanocytes.

18. The kit of claim 16, wherein the container arrangement comprises a second container including the second culture medium and the pericytes, and a third container including a third culture medium and the perivascular macrophage-type melanocytes.

19. The kit of claim 16, wherein the first culture medium comprises endothelial basal medium, fetal bovine serum, epidermal growth factor, basic fibroblast growth factor, insulin-like growth factor, vascular endothelial growth factor, ascorbic acid, hydrocortisone, and penicillin/streptomycin.

20. The kit of claim 16, wherein the second culture medium comprises Dulbecco's modified eagle's medium, fetal bovine serum, pericyte growth supplement, pigment epithelium-derived factor, and penicillin/streptomycin solution.

21. The kit of claim 16, wherein the second culture medium comprises medium 254CF plus calcium chloride, fetal bovine serum, human melanocyte growth supplement, basic fibroblast growth factor, insulin, hydrocortisone, and gentamicin/amphotericin B.

22. The kit of claim 16, wherein in the device for modelling a blood labyrinth barrier of a human ear the endothelial cells are attached to the luminal side of the membrane, the pericytes are attached to the abluminal side of the membrane, and the perivascular macrophage-type melanocytes are arranged in the second fluid channel.

23. A method for preparing a device for modelling a blood labyrinth barrier of a human ear, comprising the steps of:
cutting at least one first opening in a first medical tape and at least one second opening a second medical tape using a layout mask, respectively, wherein each of the at least one first and one second opening comprises a main portion in form of an elongated rectangle prism as well as an incoming and an outgoing portion; patterning a membrane using the layout mask,
preparing a first cover sheet and a second cover sheet using the layout mask;
assembling the device by
adhering the first cover sheet with the first medical tape having the at least one first opening portion, thereby forming a first fluid channel,
adhering the second cover sheet with the second medical tape having the at least one second opening portion, thereby forming a second fluid channel, and
adhering the membrane with the first medical tape and the second medical tape; and
curing and sterilising the device.

24. The method according to claim 23, wherein the layout mask includes electronic layouts for the first and second medical tapes, the membrane, and the first and second cover sheet, wherein the membrane preferably is patterned using a laser.

25. The method according to claim 23, comprising:
perfusing a first culture medium and a second culture medium in the first fluid channel and the second fluid channel respectively,
wherein the first culture medium comprises progenitor endothelial cells, and the second culture medium comprises progenitor pericytes and perivascular macrophage-type melanocytes.

26. The method according to claim 23, wherein each of the first medical tape and the second medical tape comprises an acrylic-based adhesive.

27. The method according to claim 23, wherein each of the first cover sheet and the second cover sheet comprises glass and/or polycarbonate.
